(19) United States
(12) Patent Application Publication
(10) Pub. No.: US 2006/0116691 A1
(43) Pub. Date: Jun. 1, 2006

(54) Guide insertion device

(76) Inventor: Fabrice Bonacci, Saint-Priest (FR)

Correspondence Address: SUGHRUE MION, PLLC, 2100 PENNSYLVANIA AVENUE, N.W., SUITE 800, WASHINGTON, DC, 20037, US

(21) Appl. No.: 10/542,443

(22) PCT Filed: Jan. 15, 2004
(86) PCT No.: PCT/FR04/00074

§ 371 Date: Jul. 15, 2005

(30) Foreign Application Priority Data
Jan. 17, 2003 (FR) 03/00527

(51) Int. Cl. A61F 11/00

(52) U.S. Cl. 606/108

(57) ABSTRACT

The guide insertion device (8) for introduction of a guide into a check valve is made from a surgical material, comprising a body (10), defining a guide duct (12), said duct being open at the two ends thereof. The duct has a fine cannula (14) extending from a first duct end along the axis of the duct, suitable for use through the stop valve. The body (10) has a groove (24) extending longitudinally along the length of the duct (12) from one end to the other, opening into the duct (12) over the whole length thereof. The above finds application for the placement of a guide in the body of a patient.

The present invention relates to a guide insertion device, of the type suitable for the insertion of a guide into a check valve made of a surgical material comprising a body delimiting a duct for guiding the guide, the duct being open at its two ends, the body having, from a first end of the duct, a slim cannula extending along the axis of the duct and suitable for being engaged through the check valve.

A guide insertion device is used in cardiology and radiology or in surgical imaging particularly for surgical cardiology procedures or radiology medical procedures.

A guide insertion device is used to insert a very flexible surgical guide into the bloodstream. The guide is formed of a very flexible steel wire having a length that can be up to two meters. The wire is formed of braided steel strands and is, where necessary, coated with a plastic sleeve.

The guide is intended to form a support for the insertion, the movement and the use of different surgical tools, such as catheters, air bladders and stents.

For the positioning of the guide, a catheter is first inserted into a vein or an artery of the patient. To prevent a flow of blood, the catheter is furnished at its end situated outside the patient with a sealed check valve making it possible to insert various tools into the body of the patient from the catheter. This valve is usually formed in a connector having a shunt. Such a connector fitted with a valve is known as a Y connector. The valve is formed of a diaphragm made of deformable rubber.

A guide insertion device is used to open the valve and pass the guide through the valve. The guide is actually too flexible to be engaged alone through the valve.

The currently known guide insertion devices comprise a body delimiting a duct through which the guide passes. The body has at one end of the duct a cannula that can be inserted into the valve to allow the guide to pass through.

Guide insertion devices are formed for example of a tube made of stainless steel or of plastic forming the cannula at one end of which an end piece made of plastic is fitted by bonding or overmolding.

For positioning it, the guide is inserted into the duct of the guide insertion device through one end of the duct passing through the end piece. The cannula of the guide insertion device is inserted through the valve. The guide is then progressively pushed into the catheter then into the bloodstream of the patient.

The guide insertion device is finally removed to allow the tools to pass along the guide. The guide insertion device is removed by causing the guide insertion device to travel along the whole length of the portion of the guide insertion device that has not been inserted into the catheter.

This maneuver is relatively tricky and risks damaging the guide which is extremely fragile.

The object of the invention is to propose a guide insertion device that can be easily removed from the guide without risk of damaging the latter.

Accordingly, the subject of the invention is a guide insertion device of the aforementioned type, characterized in that the body has a slot extending longitudinally along the length of the duct from one end to the other of this duct and opening into the duct along its whole length.

According to particular embodiments, the guide insertion device comprises one or more of the following features:
the body is formed of a single monobloc piece;
the cannula extends over a length 50% greater than the total length of the duct;
the outer surface of the cannula is generally frustoconical and flares progressively from said first end of the duct;
the body comprises around the second end of the duct a divergent frustoconical wall axially extending said cannula;
the diameter of the duct along the length of the body increases progressively from the first end to the second end;
the minimal diameter of the duct lies between 0.30 mm and 1 mm;
the width of the slot lies between 0.15 mm and 0.50 mm;
the ratio of the minimal diameter of the duct to the width of the slot lies between 5 and 9;
the length of the duct lies between 7 cm and 13 cm and
the body has two internal bevels made along the whole length of the slot between each of the opposite side walls of the slot and the surface of the duct.

The invention will be better understood on reading the following description, given only as an example and made with reference to the drawings in which:

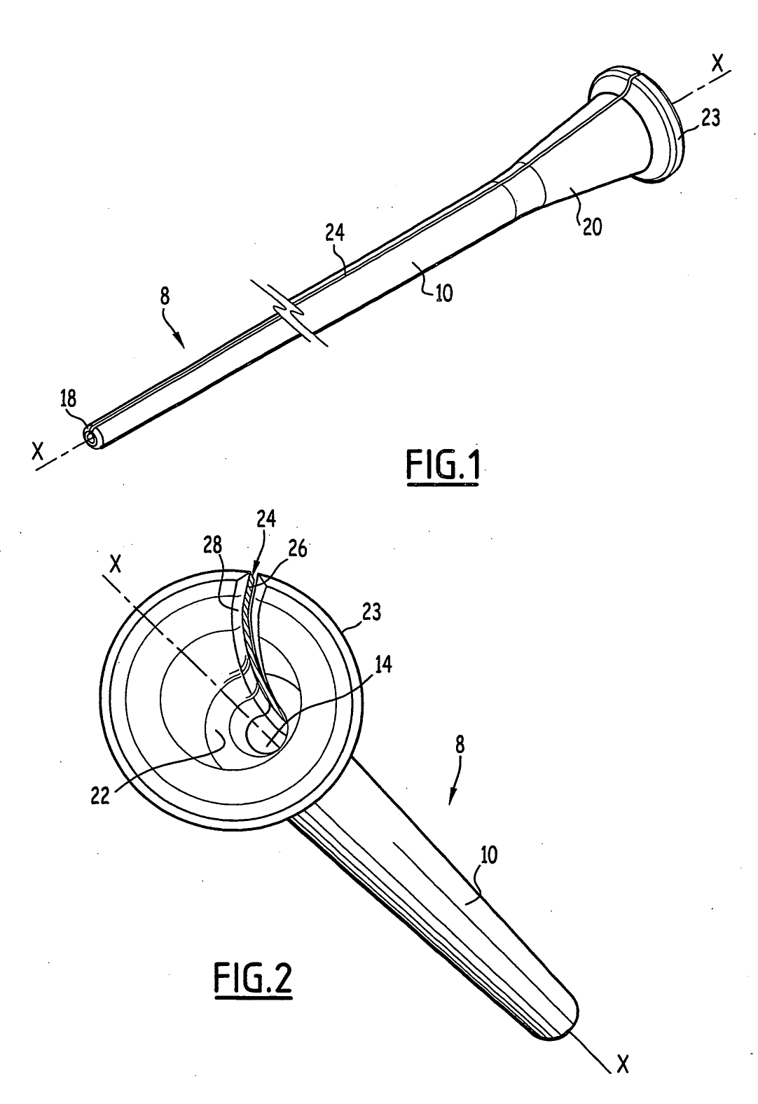

FIG. 1 is a front three-quarter view in perspective of a guide insertion device according to the invention;

FIG. 2 is a rear three-quarter view in perspective of the guide insertion device according to the invention;

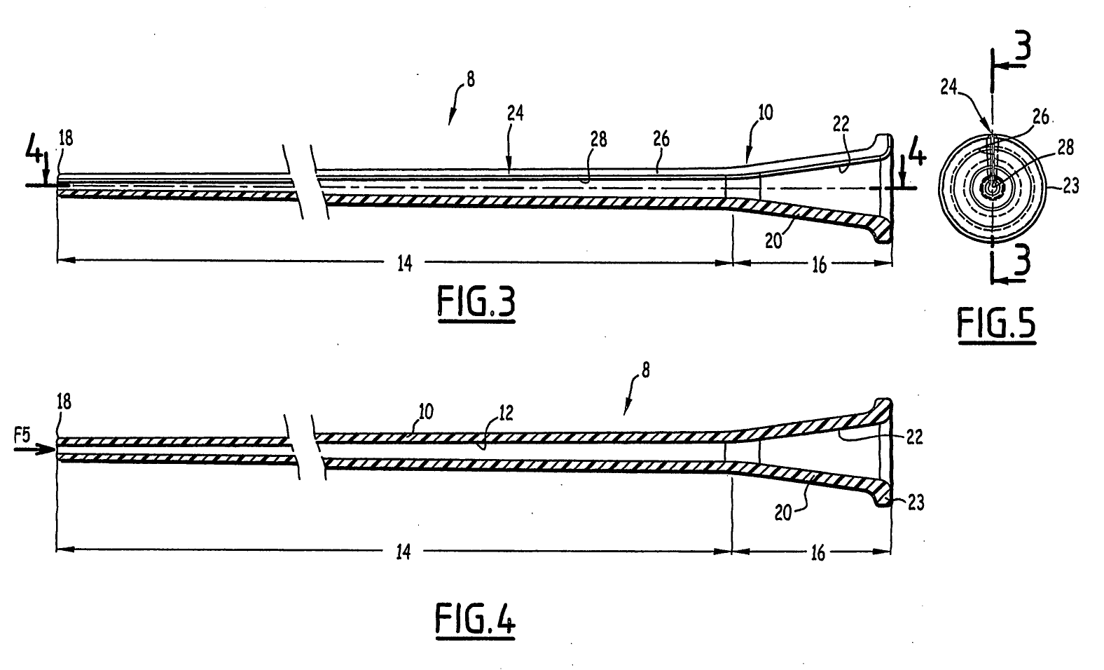

FIGS. 3 and 4 are views in longitudinal section along two planes perpendicular to one another of the guide insertion device according to the invention;

FIG. 5 is an end view of the guide insertion device taken along the arrow F5 of FIG. 4; and

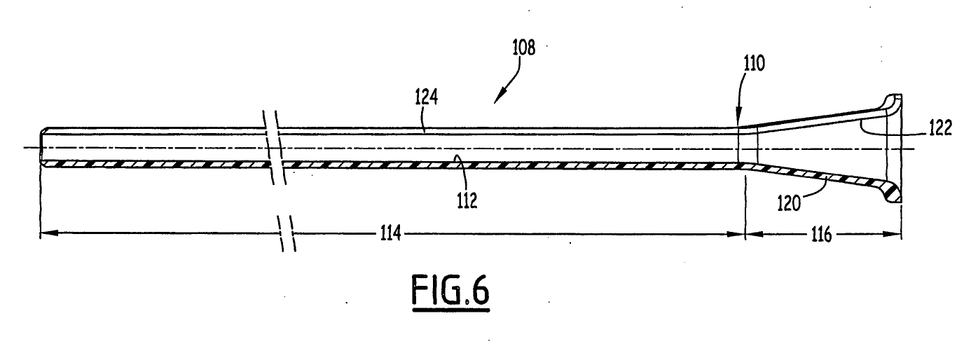

FIG. 6 is a view identical to that of FIG. 3 of a variant embodiment of a guide insertion device according to the invention.

The guide insertion device 8 shown in the figures is intended for the passage of a guide whose diameter lies between 0.010 inch and 0.023 inch, that is to say lying between 0.25 mm and 0.58 mm.

The guide insertion device 8 is formed of a monobloc body 10, that is to say that it is made in a single piece. The body is made of injected plastic.

The body of the guide is preferably made of a colored material. It is advantageously colored yellow in order to prevent its loss.

The body 10 delimits a duct 12 passing through the body from one end to the other and open at its two ends.

The body 10 is elongated and has a length measured along the axis X-X of the duct 12 lying between 7 cm and 15 cm and for example equal to 106 mm in the embodiment in question.

Along the axis X-X the body 10 has a main section 14 forming a cannula extended by a section 16 for centering the guide and for holding the guide insertion device as illustrated in FIGS. 3 and 4.

The cannula 14 extends over at least 50% of the length of the duct 12. It has a length, for example, equal to 94 mm. The cannula 14 has a circular circumference in cross section. Externally it has a diameter that increases progressively from its free end to its end connecting to the section 16. Its outer diameter varies from 1.7 mm at its free end to approximately 3 mm at its end connecting to the section 16.

Thus, the cannula has a generally frustoconical external surface.

Along the length of the cannula 14, the duct 12 flares progressively from the free end of the cannula to its connecting end. The minimal diameter of the duct lies preferably between 0.3 mm and 1 mm. This diameter varies for example from 0.65 mm to 1.50 mm. Thus, along the whole length of the cannula, the thickness of the generally frustoconical wall of the cannula is substantially constant.

Externally, at its free end, the cannula has a bead 18 making it easier to insert into the valve.

The centering and holding section 16 has a length of approximately 12 mm. This section is delimited by a generally frustoconical wall 20 extending the cannula 14 along the axis X-X of the duct. The inclination of the frustoconical wall 20 relative to the axis X-X is greater than the inclination of the wall delimiting the cannula 14.

Thus, for example, the internal diameter of the duct 12 varies along the length of the section 16 from 1.5 mm to 4.5 mm. Thus, the internal surface of the frustoconical wall 16 forms a surface that diverges towards its open end constituting a cone 22 for centering the guide when it is inserted. At its open end, the section 16 has an external peripheral collar 23 making it easier to hold the guide insertion device.

This collar has a diameter of 8 mm.

Furthermore, the body 10 of the guide insertion device has a slot 24 formed along the whole length of the duct 12 and opening into this duct.

The slot 24 thus passes through the whole thickness of the body 10. The slot is rectilinear and is made in a plane passing through the axis X-X of the duct. The width of the slot lies between 0.15 mm and 0.50 mm. In the example in question, the width is equal to 0.25 mm. Preferably, the width of the slot is less than the minimal diameter of the duct 12. Advantageously, the ratio of the minimal diameter of the duct over the width of the slot 24 lies between 5 and 9. It is substantially equal to 7 in the envisaged embodiment.

As illustrated in the figures, the side walls numbered 26 of the body delimiting the slot extend parallel to one another. These side walls link to the surface of the duct 12 via internal bevels 28 made along the whole length of the duct.

The guide insertion device according to the invention is used as follows.

The guide insertion device is held with the aid of a single hand from the holding section 16. The guide is inserted into the guide insertion device for example by engaging the end of the guide in the centering cone 22 defined by the wider portion of the duct in the section 16 of the guide insertion device. The cannula of the guide insertion device is then engaged in the valve whose diaphragm is deformed by the passage of the cannula. The guide is then pushed progressively into the catheter then into the body of the patient by sliding in the duct of the guide insertion device. The minimal diameter of the duct being greater than the diameter of the guide, this sliding process is easily carried out.

When the guide is in place, the guide insertion device is removed from the valve by traveling a short distance along the length of the guide. The guide insertion device is then removed laterally from the guide, by being disengaged laterally, the guide passing through the slot 24. Preferably, the guide insertion device is pulled transversely to the guide from its end supporting the collar, so that the guide insertion device is disengaged progressively from the guide first by the section 16 then by the cannula.

It is understood that the procedure to remove the guide insertion device may be carried out with a single hand.

The presence of the bevels 28 prevents the guide being damaged when the guide insertion device is removed. The presence of the slot 24 on the guide insertion device also makes it possible to reposition the guide insertion device when a movement of the guide becomes necessary while the guide is still engaged through the slot.

FIG. 6 shows a variant embodiment of a guide insertion device according to the invention. This guide insertion device has a shape generally identical to the guide insertion device previously described and differs therefrom by these dimensions.

Thus, the portions identical or corresponding to those of the previous guide insertion device are indicated by the same reference numbers plus 100.

The guide insertion device 108 is also intended for the passage of a guide with a diameter lying between 0.25 and 0.58 mm. Its body 110 delimits a duct 112 with a diameter greater than the diameter of the duct 12 of the previous guide insertion device. The total length of the body 110 is 105 mm.

The body consists of a main section 114 forming a cannula extended by a section 116 for centering the guide and holding the guide insertion device.

The cannula 114, 94 mm long in the example in question, and extends over at least 50% of the length of the duct 112. Its circumference is circular. Its external diameter increases progressively, the latter varying from 2.8 to 3 mm at its end connecting to the section 116.

Along the length of the cannula 114, the duct 112 flares progressively from its free end of the cannula to its connecting end. In this embodiment, the minimal diameter of the duct 112 lies preferably between 1.5 and 2.5 mm. Its diameter varies for example from 2 mm to 2.2 mm along the length of the cannula 114. Thus, the maximal diameter of the duct 112 at the end of the cannula 114 lies preferably between 1.05 and 2.4 times the minimal diameter of the duct 112.

The centering and holding section 116 delimited by a generally frustoconical wall 120 extending the cannula 114 delimits a centering cone 122 whose diameter varies internally from 2.2 to 5.3 mm. More generally, the frustoconical wall 120 internally delimits a centering cone 122 whose maximal diameter lies between 1.5 and 5 times its minimal diameter. Furthermore, advantageously, the maximal diameter of the centering cone 114 lies between 3 and 8 times the minimal diameter of the duct 112.

The slot 124 formed over the whole length of the duct has a width lying in this embodiment between 0.08 and 0.15 mm. It is for example 0.12 mm. Preferably, in this embodiment, the ratio of the minimal diameter of the duct 112 to the width of the slot 124 lies between 12 and 22. In the embodiment shown, this ratio is substantially equal to 17.

In this embodiment, the internal diameter of the duct 112, at least along the length of the cannula 114, is greater than the corresponding diameter of the duct of the guide insertion device of the previous embodiment. Thus, the guide insertion device is easier to produce.

Advantageously, ribs, for example four in number, are provided on the outer surface of the generally frustoconical wall 120 to make the guide insertion device easier to hold.

1. A guide insertion device (8; 108) suitable for the insertion of a guide into a check valve made of a surgical material comprising a body (10; 110) delimiting a duct (12; 112) for guiding the guide, the duct being open at its two ends, the body having, from a first end of the duct, a slim cannula (14; 114) extending along the axis of the duct and suitable for being engaged through the check valve, characterized in that the body (10; 110) has a slot (24; 124) extending longitudinally along the length of the duct (12; 112) from one end to the other of this duct and opening into the duct (12; 112) along its whole length, in that the diameter of the duct (12; 112) along the length of the body (10; 110) increases progressively from the first end to the second end, and in that the outer surface of the cannula (14; 114) is generally frustoconical and flares progressively from said first end of the duct.

2. The guide insertion device as claimed in claim 1, characterized in that the body (10; 110) is formed of a single monobloc piece.

3. The guide insertion device as claimed in claim 1, characterized in that the cannula (14; 114) extends over a length 50% greater than the total length of the duct (12; 112).

4. The guide insertion device as claimed in claim 1, characterized in that the body (10; 110) comprises around the second end of the duct (12; 112) a divergent frustoconical wall (20; 120) axially extending said cannula (14; 114).

5. The guide insertion device as claimed in claim 4, characterized in that said frustoconical wall (20; 120) internally delimits a centering cone (22; 122) whose maximal diameter lies between 1.5 and five times its minimal diameter.

6. The guide insertion device as claimed in claim 4, characterized in that said frustoconical wall (20; 120) internally delimits a centering cone (22; 122) whose maximal diameter lies between 3 and 8 times the minimal diameter of the duct (12; 112).

7. The guide insertion device as claimed in claim 1, characterized in that the minimal diameter of the duct (112) lies between 1.5 mm and 2.5 mm.

8. The guide insertion device as claimed in claim 1, characterized in that the width of the slot (124) lies between 0.08 and 0.15 mm.

9. The guide insertion device as claimed in claim 1, characterized in that the ratio of the minimal diameter of the duct (112) to the width of the slot (124) lies between 12 and 22.

10. The guide insertion device as claimed in claim 1, characterized in that the minimal diameter of the duct (12) lies between 0.30 mm and 1 mm.

11. The guide insertion device as claimed in claim 1, characterized in that the width of the slot (24) lies between 0.15 mm and 0.50 mm.

12. The guide insertion device as claimed in claim 1, characterized in that the ratio of the minimal diameter of the duct (12) to the width of the slot (24) lies between 5 and 9.

13. The guide insertion device as claimed in claim 1, characterized in that the length of the duct (12; 112) lies between 7 cm and 13 cm.

14. The guide insertion device as claimed in claim 1, characterized in that the body (10; 110) has two internal bevels (28) made along the whole length of the slot (24; 124) between each of the opposite side walls of the slot (24; 124) and the surface of the duct (12; 112).
